(19) United States
(12) Patent Application Publication
(10) Pub. No.: US 2005/0232977 A1
(43) Pub. Date: Oct. 20, 2005

(54) Metered mixing technology for improved taste masking

(76) Inventors: Sadath U. Khan, Randolph, NJ (US); Neema M. Kulkarni, Randolph, NJ (US); Narendra Vutla, Kendall Park, NJ (US)

Correspondence Address: Warner-Lambert Company LLC, 201 Tabor Road, Morris Plains, NJ, 07960, US

(21) Appl. No.: 11/110,590

(22) Filed: Apr. 20, 2005

Related U.S. Application Data
(60) Provisional application No. 60/563,611, filed on Apr. 20, 2004.

(51) Int. Cl. A61K 9/16; A61K 9/50

(52) U.S. Cl. 424/443; 264/109

(57) ABSTRACT

The present invention relates to metered mixing technology for producing a film type product having improved taste-masking properties of a drug substance. In one aspect, a film-based product and a process for making the product are provided wherein a drug substance coated with a taste-masking substance and a film-forming solution are suitably combined and mixed yet the contact time between the components is controlled such that a bitter taste quality is not sensed by a person ingesting the product.

This application claims priority to Provisional Patent Application 60/563,611, filed Apr. 20, 2004.

### FIELD OF THE INVENTION

The present invention relates to fast-dissolving, orally consumable films, which can be used to deliver pharmaceutical agents, breath deodorizing agents, antimicrobial agents and salivary stimulants to the oral cavity. Broadly, the invention relates to providing bitter substances, such as drugs, in combination with a taste-masking material. More particularly, a process and product suitable for delivering taste-masked drug substances in an instantaneous-release, film dosage form are described.

### DESCRIPTION OF RELATED ART

It is known that many drug substances have objectionable taste characteristics that occur upon ingestion and create an unpleasant aftertaste. Although there have been attempts to produce and incorporate materials for masking the unfavorable taste of these drug substances, none have been successful in producing a film-based product with desirable taste qualities due to the nature of the manufacturing process for film-based products. The known process involves mixing a drug substance and taste-masking material in a vessel and dispensing the mixture to form film-based products. By its nature, this process often produces bitter-tasting products.

### SUMMARY

The invention relates to providing a physiologically acceptable film, which is particularly well adapted to adhere to and rapidly dissolve in the mouth of a consumer without the negative consequences of a bitter aftertaste. In one aspect, the invention provides a product and a process for manufacturing an instantaneous-release, film-based drug product that does not result in the detection of an unpleasant taste by the person taking the drug product. It has been found that the effectiveness of taste-masking materials to conceal the unpleasant taste associated with certain drug substances is related to the amount of time that the taste-masked drug substance is in contact with the film-forming solution prior to casting and drying. That is, the bitter taste associated with instantaneous-release, film-based products may be minimized or eliminated by reducing the amount of time that the taste-masked drug substance is exposed to the film-forming solution. In one aspect, a film-based product and a process for making the product are provided wherein the contact time between a drug substance coated with a taste-masking substance and a film-forming solution is controlled and is reduced such that a bitter taste quality is not sensed or is minimized by a person ingesting the product. Suitable film products include a single-layer or multiple-layer film.

### BRIEF DESCRIPTION OF THE DRAWINGS

FIG. 1 is a view of a system for making a consumable film.

### DETAILED DESCRIPTION

An aspect of the invention is directed to a method for producing a supple, non-self-adhering film especially suitable for oral delivery. Suitable films include standalone single layer films and multiple layer films.

An embodiment of the present invention provides a method for making a consumable film including the steps of adding a film-forming solution to an extrusion device; adding a drug substance to the extrusion device, said drug substance including a taste-masking substance; mixing the film-forming solution and the taste-masked drug substance to form a mixture in the extrusion device; extruding the mixture on a substrate; and drying the mixture to provide the consumable film. Another embodiment of the present invention provides a multiple layer film produced by the aforementioned method including coextruding two films produced by the aforementioned method. A further embodiment of the present invention provides a consumable film produced according a method of the present invention, wherein the film adheres to and dissolves in a mouth of a consumer, wherein the amount of contact time between the film-forming solution and the taste-masked drug substance is controlled during processing such that a bitter aftertaste is eliminated when the film is ingested.

In one aspect, a film and a process for making the film product are provided wherein a taste-masked active pharmaceutical ingredient and a film-forming solution are suitably or sufficiently combined and mixed yet the contact time between the components is controlled such that a bitter taste quality is not sensed by a person ingesting the product.

Another embodiment of the present invention provides a method for making a consumable film including the steps of adding a film-forming solution to a mixing device; adding a taste-masked drug substance; mixing the film-forming solution and the taste-masked drug substance to form a mixture for a mixing time; casting the mixture on a substrate; and drying the mixture to provide the consumable film; wherein the mixing time of the film-forming solution and the taste-masked drug substance is less than about thirty minutes. A taste-masked drug substance includes an active pharmaceutical ingredient or drug substance and a taste masking agent. Suitable taste masking techniques include partial or full coatings, partial or full encapsulation, partial or full adsorption complexes and the like and combinations thereof. Useful ratios of drug or active to taste masking agent include from about 10:1 to about 1:10, from about 3:1 to about 1:3 and from about 1:1.

Another embodiment of the present invention provides a method for making a consumable film, including adding a film-forming solution to a mixing device; adding a drug substance to the mixing device, said drug substance including a taste-masking substance; mixing the film-forming solution and the taste-masked drug substance to form a mixture for an amount of contact time; controlling the amount of contact time between the film-forming solution and the taste-masked drug substance; casting the mixture on a substrate; and drying the mixture to provide the consumable film; wherein the amount of contact time is such that the consumable film does not have a bitter taste.

In another embodiment, a method is provided including the steps of mixing a film-forming agent and at least one stabilizing agent to provide a film-forming mixture; dissolving water-soluble ingredients, such as taste-masked drug substances, in water to provide an aqueous solution; combining the film-forming mixture and the aqueous solution to provide a hydrated polymer gel; mixing oils to form an oil mixture; adding the oil mixture to the hydrated polymer gel and mixing to provide a uniform emulsified gel; casting the uniform gel on a substrate; and drying the cast gel to provide a film. The uniform gel may be a single layer extruded as a film on a paper substrate that is subsequently peeled off the substrate after drying and then cut based on dosing considerations.

The film may also be referred to as a flat, foil, paper or wafer type product. Multiple-layer film may be produced by coextrusion, by casting the uniform gel on a previously cast film, by extruding a second mixture onto a previously dried film, or by adhering two previously cast films together with an adhesive. A product may provide a system for the application and release of drug and other active substances.

In another aspect, at least one drug substance and a taste-masking substance are combined with a film-forming solution to create an instantaneous-release, film-based medicinal product for the purpose of delivering a drug to the oral cavity of a human while minimizing or eliminating any unpleasant immediate or subsequent taste associated with the product. Under normal dosing conditions, after the product is placed in the mouth of the person receiving the drug, the product dissolves instantaneously thereby releasing, among other ingredients, the taste-masked drug substance. In one aspect, upon release, the taste-masking substance counteracts the unpleasant taste quality inherent to the drug substance and provides a non-bitter taste immediately post-ingestion of the product. In another aspect, the product retains its taste-masking properties while retaining bioavailable properties of the drug substance when ingested.

As discussed above, it has been determined that known processes for taste-masking a drug substance provided in a film dosage form fail to consistently retain the taste-masking properties to overcome the unpleasant taste sensed from many drug substances. It has been found that the undesirable taste quality may be a consequence of the increased contact time between the taste-masked drug substance and the film-forming solution or mixture. While not wishing to bound to any particular theory, it is believed that a film-forming solution may permeate into a taste-masked coating surrounding the drug substance over time prior to film formation, making the taste-masking substance ineffective and resulting in an unpleasant tasting product. In fact, as the contact time of the film-forming solution increases, the effectiveness of the taste-masking substance is reduced as a gradual increase in the bitterness of the film product is detected. Specifically, it has been found that if contact time is kept under approximately thirty minutes, the taste-masking substance is effective; however, as contact time increases beyond this time period, the amount of bitterness detected from the product increases. Moreover, it is useful to further reduce the contact time to fifteen minutes, ten minutes, five minutes, two minutes, 60 seconds, 30 seconds and shorter.

In addition to improved operating conditions, a technologically innovative processing technique has been developed that curtails the progressive ineffectiveness of the taste-masking substances. In one aspect, a film-forming solution and taste-masked drug substance are simultaneously fed at predetermined rates into a continuous mixing device such as an extrusion device. The rates may be determined experimentally or theoretically based on product specifications. The components are combined and subsequently processed to form a film-dosage form product without a lengthy contact time period where the composition awaits processing. Thus, the process is controlled to minimize the contact or residence time for the taste-masked drug substance and film-forming mixture in the combined solution, resulting in minimal to no loss of effectiveness of the taste-masking substance. Therefore, under these designed conditions, the taste-masking substance remains effective and the bitter tasting quality of the film-formed product is reduced or eliminated.

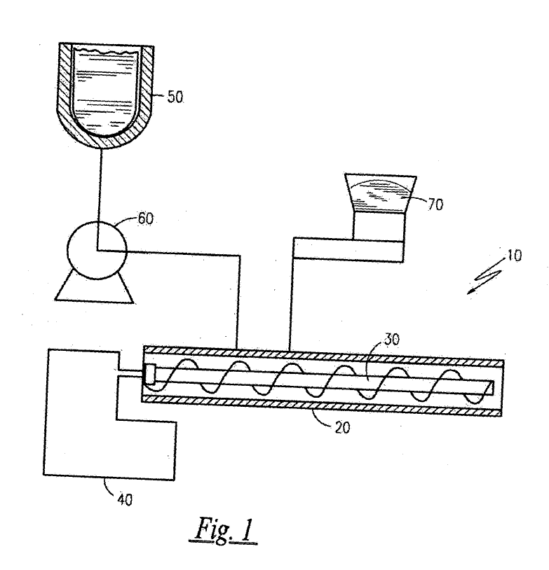

FIG. 1 provides a system 10 for producing a consumable film. In this illustrative example, liquid, such as a film-forming solution, may be transferred from a vessel 50 to an extrusion barrel 20 via pump 60. Multiple liquids may be combined and transferred or may be contained in separate, designated vessels and transferred to the extrusion barrel 20 via separate pumps (not shown). Solid materials, such as coated, taste-masked drugs in powder form, may be transferred from a hopper 70 to the extrusion barrel 20. An auger (not shown) may be employed to facilitate the solids transfer. Of course, multiple solids may be combined and transferred or may be contained in separate hoppers (not shown) and separately transferred to the extrusion barrel 20. The extrusion screw 30 may be powered by extrusion motor 40 and the feed materials may be mixed, heated (if necessary), and extruded through the end of the extrusion barrel 20 and cast onto a substrate where the film is subsequently dried and cut into dosage amounts. Multiple devices may be used to produce a multiple-layer film or the illustrated device may be employed to extrude and cast material onto a previously cast film.

Various types of manufacturing equipment, including, for example, conventional machines for performing extrusion molding, injection molding, casting, or dip molding, may be employed in any number of configurations to carry out the various processes of the invention. In one embodiment, the devices disclosed in U.S. Pat. No. 6,499,984, the disclosure of which is expressly incorporated by reference in its entirety, provide structures that are useful in practicing the invention. In one embodiment, flow meters and associated control valves are employed to regulate the metered addition of the drug substance and the contents of the film-forming solution; however, any other suitable metering and control devices may be utilized. A twin screw extruder may be utilized as a continuous mixing device, or, more specifically as described in the U.S. Pat. No. 6,499,984, a twin screw wet granulator-chopper may be employed; however, any other suitable mixing device may be used as long as the contact time between the coated drug substance and the film-forming solution is minimized in accordance with the principles of the invention.

In one aspect, a film-forming solution may be continuously fed to an extrusion device, such as a Haake twin screw mixer, from a holding or mixing vessel at a predetermined flow rate, e.g., approximately 8 gal/min. Simultaneously, a taste-masked drug substance, such as Dextromethorphan, may be continuously fed at a predetermined rate, e.g., approximately 4 gal/min, to the extrusion device for a 33% drug load. As the extruder screws are rotated, the film-forming solution and taste-masked drug substance are intimately combined, mixed, and extruded. The material may then be cast, dried, and cut. The design of the extrusion device and the predetermined and controlled flow rates of the feed materials provide controlled contact times of the materials prior to solidification.

Suitable taste masking materials include, but are not limited to, Eudragits (E100, EPO, RL, RD) sold by Rohm Pharma, Amberlite®, sold by Rohm & Haas, cellulose acetate, hydroxypropyl cellulose hydroxypropylcellulose, ethycellulose, hydroxypropylmethylcellulose (Aquacoat®), ethylcellulose, methacrylates, acrylic co-polymers such as Eudragit® (Butylmethacrylat-(2-Dimethylaminoethyl)methacrylat-Methylmethacrylat-Copolymer (1:2:1)”), KOLLICOAT®, polyvinylpyrrolidone and combinations thereof. The taste masked drug product can be in the form of a microencapsulation, ion-exchange resin complex, such as a sulfonated polymers, electro-chemical melt, supercritical fluids, magnesium trisilicate, coacervation, or cyclodextrin (cyclic-linked oligosaccharides) complexes. Useful sulphonated polymers include polystyrene cross-linked with 8% of divinylbenzene such as Amberlite®IRP-69 and IRP-64 (obtained by Rohm and Haas), Dow XYS-40010.00®, Dow XYS40013.00® (obtained from the Dow Chemical Company).

A wide variety of drug substances and water-soluble polymers may be useful for carrying out the invention, including those disclosed in U.S. Provisional Patent Application Ser. No. 60/467,339, the disclosure of which is incorporated by reference in its entirety. Lubricants and plasticizers may be added to the solutions. Film-forming solutions with suitable film forming polymers include but are not limited to pullulan, gelatins, starches, celluloses and combinations thereof may be employed. Other useful materials are disclosed in U.S. Pat. No. 6,596,298, the disclosure of which is incorporated by reference in its entirety. Useful active pharmaceutical ingredients (APIs) or drug products include antimicrobial agents, non-steroidal anti-inflammatory agents, antitussives, decongestants, anti-histamines, expectorants, anti-diaherrals, H₂-antagonists, proton pump inhibitors, analgesics, stimulants and combinations thereof. Useful APIs include diphenhydramine, dextromethorphan, phenylephrine, menthol, pseudoephedrine, acetaminophen, ibuprofen, famotidine, guaifenesin, ketoprofen, nicotine, celecoxib, valdecoxib, chlorpheniramine, fexofenadine, loratadine, desloratadine, cetirizine, ranitidine, simethicone, and isomers, pharmaceutically acceptable salts and prodrugs thereof and combinations thereof.

### EXAMPLE 1

A film-forming solution is continuously fed to an extrusion device, such as a Haake twin screw mixer, from a holding or mixing vessel at a predetermined flow rate, e.g., approximately 8 gal/min. Simultaneously, Dextromethorphan coated with Amberlite is continuously fed at a predetermined rate, e.g., approximately 4 gal/min, to the extrusion device for a 33% drug load. As the extruder screws are rotated, the film-forming solution and taste-masked drug substance are combined, mixed, and extruded. The material is then be cast, dried, and cut. The design of the extrusion device and the predetermined and controlled flow rates of the feed materials provide controlled contact times of the materials prior to solidification. The resulting product tasted good with minimal or no bitter taste.

While the invention has been described in detail and with reference to specific examples thereof, it will be apparent to one skilled in the art that various changes and modifications can be made therein without departing from the spirit and scope thereof.

1. A method for making a consumable film, comprising:
adding a film-forming solution to an extrusion device;
adding a drug substance to the extrusion device, said drug substance including a taste-masking substance;
mixing the film-forming solution and the taste-masked drug substance to form a mixture in the extrusion device;
extruding the mixture; and
drying the mixture to provide the consumable film.

2. The method of claim 1, wherein the drug substance is coated with the taste-masking substance.

3. The method of claim 1, wherein the amount of contact time between the film-forming solution and the taste-masked drug substance is less than about thirty minutes.

4. The method of claim 1, wherein the amount of contact time between the film-forming solution and the taste-masked drug substance is less than about five minutes.

5. A single-layer film produced according to claim 1.

6. A multiple-layer film produced by the method comprising coextruding two films produced by the method according to claim 1.

7. A consumable film produced according to claim 1 that adheres to and dissolves in a mouth of a consumer, wherein the amount of contact time between the film-forming solution and the taste-masked drug substance is controlled during processing such that a bitter aftertaste is eliminated when the film is ingested.

8. A method for making a consumable film, comprising:
adding a film-forming solution to a mixing device;
adding a taste-masked drug substance;
mixing the film-forming solution and the taste-masked drug substance to form a mixture for a mixing time;
casting the mixture on a substrate; and
drying the mixture to provide the consumable film;
wherein the mixing time of the film-forming solution and the taste-masked drug substance is less than about thirty minutes.

9. The method of claim 8, wherein the drug substance is coated with the taste-masking substance.

10. The method of claim 8, wherein the mixing time is less than about ten minutes.

11. A single-layer film produced according to claim 8.

12. A multiple-layer film produced by the method according to claim 8 further comprising extruding a second mixture onto a previously dried film.

13. A consumable film produced according to claim 8 that adheres to and dissolves in a mouth of a consumer, wherein the mixing time between the film-forming solution and the taste-masked drug substance is minimized during processing such that a bitter aftertaste is eliminated when the film is ingested.

14. A method for making a consumable film, comprising:
adding a film-forming solution to a mixing device;
adding a drug substance to the mixing device, said drug substance including a taste-masking substance;
mixing the film-forming solution and the taste-masked drug substance to form a mixture for an amount of contact time;
controlling the amount of contact time between the film-forming solution and the taste-masked drug substance;
casting the mixture on a substrate; and
drying the mixture to provide the consumable film;
wherein the amount of contact time is such that the consumable film does not have a bitter taste.

15. The method of claim 14, wherein the drug substance is coated with the taste-masking substance.

16. The method of claim 14, wherein the amount of contact time is less than about thirty minutes.

17. The method of claim 14, wherein the amount of contact time is less than about five minutes.

18. A single-layer film produced according to claim 14.

19. A multiple-layer film produced by the method comprising adhering two films produced by the method of claim 14 together with an adhesive.

20. A consumable film produced according to claim 14 that adheres to and dissolves in a mouth of a consumer, wherein the contact time between the film-forming solution and the taste-masked drug substance is controlled during processing such that a bitter aftertaste is eliminated when the film is ingested.
